(19) United States
(12) Patent Application Publication
(10) Pub. No.: US 2009/0298891 A1
(43) Pub. Date: Dec. 3, 2009

(54) Methods of Treating or Preventing Cancer Using Pyridine Carboxaldehyde Pyridine Thiosemicarbazone Radiosensitizing Agents

(75) Inventors: Philip Tofilon, Tampa, FL (US); Kevin Camphausen, McLean, VA (US); David Gius, Clarksville, MD (US)

Correspondence Address: EDWARDS ANGELL PALMER & DODGE LLP, PO BOX 55874, BOSTON, MA, 02205, US

(73) Assignee: GOVERNMENT OF THE US, AS REPRESENTED BY THE SECRETADY OF HEALTH AND HUMAN SERVICES, Rockville, MD (US)

(21) Appl. No.: 11/992,002

(22) PCT Filed: Sep. 15, 2006
(86) PCT No.: PCT/US2006/036044

§ 371 Date: Aug. 13, 2009

Related U.S. Application Data
(60) Provisional application No. 60/718,172, filed on Sep. 16, 2005.

(51) Int. Cl. A61K 31/44; C12N 13/00; C07D 213/75; C07D 213/73

(52) U.S. Cl. 514/353; 435/173.1; 546/306

(57) ABSTRACT

The present invention features methods of inhibiting ribonucleotide reductase and DNA synthesis after administration of a dose of ionizing radiation to cells. The present invention further features methods of treating patients suffering from cancer comprising contemporaneous or sequential administration of a radiosensitizing dose of a 2-carboxyaldehyde pyridine thiosemicarbazone compound and ionizing radiation.

### RELATED APPLICATIONS

This application claims priority to U.S. Provisional Application No. 60/718,172, filed 16 Sep. 2005, the content of which is hereby incorporated by reference in its entirety.

### GOVERNMENT SUPPORT

This work described herein was supported by the National Institutes of Health. The U.S. Government may have certain rights in the invention.

### BACKGROUND OF THE INVENTION

1. Field of the Invention

The present invention provides methods of treating patients suffering from or susceptible to cancer, methods of retarding or reversing tumor growth, and methods of inhibiting DNA synthesis in cells by co-administration of a radiosensitizing amount of a 2-carboxyaldehyde pyridine thiosemicarbazone compound and at least one dose of ionizing radiation, for example a radiation dose sufficient to cleave main chains of DNA double helix.

2. Background.

Although the molecular events determining cell survival after exposure to ionizing radiation have not been completely defined, it is clear that critical processes in determining radioresponse include DNA repair, cell cycle checkpoint activation and apoptosis. Because these events are under the control of a variety of signaling pathways, it is apparent that multiple independent and interacting processes can regulate radiation-induced cell death. In an evaluation of malignant and normal cells, Slupianek et al showed that, in response to DNA-damaging drugs, leukemic cells had enhanced DNA repair capability, prolonged checkpoint activation and increased resistance to apoptosis as compared to their normal cell counterpart (Slupianek A, Hoser G, Majsterek I, Bronisz A, Malecki M, Blasiak J. et al. Fusion tyrosine kinases induce drug resistance by stimulation of homology-dependent recombination repair, prolongation of G (2)/M phase, and protection from apoptosis. Molecular and Cellular Biology 2002; 22:4189-201). These data are consistent with the existence of multiple drug resistance mechanisms in leukemic cells (Skorski T. BCR/ABL regulates response to DNA damage: the role in resistance to genotoxic treatment and in genomic instability, Oncogene 2002; 21:8591-604). Extrapolation of these results to solid tumor cells would suggest that multiple and possibly redundant processes contribute to the resistance of tumor cells to radiation.

Current efforts to develop strategies for enhancing tumor radiosensitivity have focused on the use of agents that target a single molecule putatively involved in regulating radiation-induced cell death. However, assuming that a combinatorial process determines radiosensitivity, a more effective approach would be to target multiple radioresponse regulatory molecules. Moreover, the ability of a specific molecule to affect radioresponse often depends on the genetic background of the tumor cell. For example, p53 (Slichenmyer W, Nelson W, Slebos R, Kastan M. Loss of a p53-associated G1 checkpoint does not decrease cell survival following DNA damage. Cancer Research 1993; 53:4164-8; and Bristow R, Benchimol S, Hill R. The p53 gene as a modifier of intrinsic radiosensitivity: implications for radiotherapy. Radiotherapy and Oncology 1996; 40:197-223), Chk1 (Koniaras K, Cuddihy A, Christopoulos H, Hogg A, O'Connell M. Inhibition of Chk1-dependent G2 DNA damage checkpoint radiosensitizes p53 mutant human cells. Oncogene 2001; 20:7453-6) and NFκB (Russell J, Tofilon P. Radiation-induced activation of nuclear factor-kappaB involves selective degradation of plasma membrane-associated I(kappa)B(alpha). Molecular and Cellular Biology 2002; 13:3431-40) have been shown to influence the radiosensitivity of some but not all tumor cells.

Thus, it would be desirable to provide new therapeutic protocols for treatment of cancer and tumors in which a therapeutic agent possess greater radiosensitization ability by inhibiting one or more radioresponse regulatory processes. In particular, it would be desirable to provide improved radiological therapeutic protocols which reduce the percentage of viable tumor cells post-irradiation.

### SUMMARY OF THE INVENTION

The present invention provides methods of preventing DNA synthesis and DNA repair after exposing cells to ionizing radiation by contacting the irradiated cells with a 2-carboxyaldehyde pyridine thiosemicatbazone compound before or after irradiation of the cell. The present invention further provides methods of treating or preventing cancer and other tumors by coadministration of a radiosensitizing amount of a 2-carboxyaldehyde pyridine thiosemicarbazone compound and ionizing radiation. The present invention further provides assays for identifying radiosensitization agents which provide enhanced cell death in tumors, carcinomas, and related malignant tissues.

In certain aspects, the invention provides a method for inhibiting DNA synthesis and DNA repair in a cell comprising the steps of: (1) providing a 2-carboxyaldehyde pyridine thiosemicarbazone compound or prodrug thereof; (2) contacting the cell with a radiosensitizing amount of the 2-carboxyaldehyde pyridine thiosemicarbazone compound or prodrug thereof; and (3) exposing the cell to ionizing radiation sufficient to induce double strand breaks in DNA of the cell. In certain methods the step of contacting the cell with a radiosensitizing amount of the 2-carboxyaldehyde pyridine thiosemicarbazone compound may occur before, after, or contemporaneously with exposure of the cell to ionizing radiation.

In certain other aspects, the invention provides a method of treating a patient suffering from or susceptible to cancer, the method comprising the steps of: (1) providing a 2-carboxyaldehyde pyridine thiosemicarbazone compound or prodrug thereof; (2) administering to the patient a radiosensitizing amount of the 2-carboxyaldehyde pyridine thiosemicarbazone compound or prodrug thereof; and (3) administering a dose of ionizing radiation. In certain therapeutic methods the dose of ionizing radiation is sufficient to induce double strand DNA cleavage. In certain other therapeutic methods, the step of administering the radiosensitizing amount of the 2-carboxyaldehyde pyridine thiosemicarbazone compound to the patient may occur before, after, or contemporaneously with administering the dose of ionizing radiation to the patient.

Other aspects of the invention are described infra.

### BRIEF DESCRIPTION OF THE DRAWINGS

FIG. 1 is a logarithmic plot of surviving fraction from a clonogenic assay of Triapine (3-amino-2-carboxaldehyde-pyridine thiosemicarbazone) administered to PSN1 (pancreatic carcinoma) cell line in which “control” is the administration of ionizing radiation without Triapine, “pre-IR” represents cell survival when Triapine (3 μM) is administered prior to ionizing radiation, and “post-IR” represents cell survival when Triapine (3 μM) is administered after ionizing radiation;

FIG. 2 is a logarithmic plot of surviving fraction from a clonogenic assay of triap Triapine ine administered to U251 (human glioma) cell line in which “control” is the administration of ionizing radiation without Triapine, “pre-IR” represents cell survival when Triapine (5 μM) is administered prior to ionizing radiation, and “post-IR” represents cell survival when Triapine (5 μM) is administered after ionizing radiation;

FIG. 3 is a logarithmic plot of surviving fraction from a clonogenic assay of Triapinie administered to DU145 (prostate carcinoma) cell line in which “control” is the administration of ionizing radiation without Triapine, “pre-IR” represents cell survival when Triapine (5 μM) is administered prior to ionizing radiation, and “post-IR” represents cell survival when Triapine (5 μM) is administered after ionizing radiation;

FIG. 4 is a logarithmic plot of surviving fraction from a clonogenic assay of Triapine administered to MRC5 (normal human fibroblast) cell line in which “control” is the administration of ionizing radiation without Triapine, “pre-IR” represents cell survival when Triapine (5 μM) is administered prior to ionizing radiation, and “post-IR” represents cell survival when Triapine (5 μM) is administered after ionizing radiation;

FIG. 5 is a bar graph of percentage of U251 cells in various cycle phases when the cells are suspended in dimethylsulfoxide solutions with or without Triapine (5 μM);

FIG. 6 is a time plot of DNA synthesis activity in U251 cells after administration of Triapine or hydroxylurea;

FIG. 7 is a plot of γH2aX foci per cell at various time points after irradiation;

FIG. 8 is a plot of U251 tumor volume at Day 0-40 after administration of Triapine alone, radiation alone, or co-administration of Triapine and radiation; and

FIG. 9 is a plot of PSN1 tumor volume at Day 0-40 after administration of Triapine alone, radiation alone, or co-administration of Triapine and radiation.

### DETAILED DESCRIPTION OF THE INVENTION

The present invention provides methods of inhibiting ribonucleotide reductase activity, an essential enzyme in DNA synthesis and DNA repair pathways. The invention further provides methods of preventing DNA synthesis and DNA repair and methods of treating patients suffering from cancer. The methods of the invention comprise co-administration of a radiosensitizing amount of a 2-carboxyaldehyde pyridine thiosemicarbazone compound and a dose of ionizing radiation sufficient to induce double strand cleavage of DNA in irradiated cells contacted with the 2-carboxyaldehyde pyridine thiosemicarbazone compound.

In certain methods of inhibiting DNA synthesis and/or DNA repair in a cell, the cells and the 2-carboxyaldehyde-pyridine thiosemicarbazone compound are contacted in vitro. In certain other methods the cells and the 2-carboxyaldehyde-pyridine thiosemicarbazone compound are contacted in vivo.

In certain aspects, the methods of inhibiting DNA synthesis and/or DNA repair in a cell include the administration of a 2-carboxyaldehyde pyridine thiosemicarbazone compound selected from compounds of Formula I:

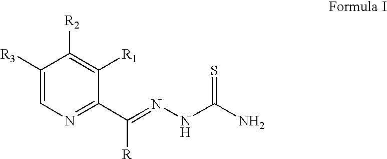

wherein

R₁ is NHR₄ or NR₄R₅, and R₃ is hydrogen; or

R₁ is hydrogen and R₃ is NHR₄, NR₄R₅ or OH;

R and R₂ are independently selected from hydrogen and C₁-C₄alkyl

R₄ is hydrogen, hydroxyl, or C₁-C₄alkyl; and

R₅ is C₁-C₄alkyl; or a pharmaceutically acceptable salt or hydrate thereof.

In certain methods of inhibiting DNA synthesis and/or DNA repair, the compound of Formula I is selected from those in which R₁ is NHR₄ or NR₄R₅; and R₃ is hydrogen. In certain other methods, the compound of Formula I is selected from those compounds in which R₁ is NH₂ and R₃ is hydrogen. In certain other methods, the compound of Formula I is selected from those compounds in which R is hydrogen. In certain other methods, a compound of Formula I is administered in which R₂ is hydrogen or methyl.

In yet other methods of inhibiting DNA synthesis or DNA repair, the compound of Formula I is selected from those compounds in which R and R₂ are independently selected from hydrogen and methyl, R₁ is amino (NH₂), and R₃ is hydrogen. Certain compounds of Formula I which are suitable for use in the methods of inhibiting DNA synthesis or DNA repair include 3-amino-2-carboxyaldehyde pyridine thiosemicarbazone and 4-methyl-3-amino-2-carboxyaldehyde pyridine thiosemicarbazone, and salts or hydrates, thereof.

In certain other aspects, the methods of inhibiting DNA synthesis and/or DNA repair in a cell include the administration of a prodrug of a 2-carboxyaldehyde pyridine thiosemicarbazone compound of Formula I, e.g., a compounds of Formula II:

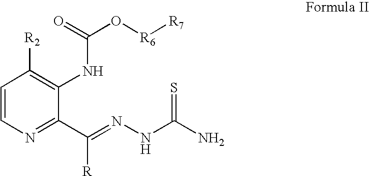

wherein

R₂ is hydrogen or methyl;

R₆ is CHR, benzyl, or ortho- or para-substituted benzyl;

R is hydrogen or C₁₋₃alkyl;

R₇ is a free acid phosphate, phosphate salt or a —S—S—R₈ residue;

R₈ is CH₂CH₂NHR₉, CH₂CH₂OH, CH₂CH₂COOR₁₀, ortho- or para-substituted phenylC₁₋₃alkyl, or ortho- or para-nitrophenyl;

R₉ is hydrogen, C₁₋₄alkanoyl, trifluoroacetyl, benzoyl, or substituted benzoyl;

R₁₀ is hydrogen, C₁₋₄alkyl, phenyl, substituted phenyl, benzyl, or substituted benzyl.

In certain methods of inhibiting DNA synthesis and/or DNA repair in a cell the dose of ionizing radiation administered to the cell is between about 0.01 Gy to about 100 Gy, between about 0.5 Gy to about 50 Gy, or between about 1 Gy to about 20 Gy for each dose.

In certain other methods of inhibiting DNA synthesis and/or DNA repair in a cell, the cell is exposed to a protocol in which the 2-carboxyaldehyde-pyridine thiosemicarbazone and ionizing radiation are co-administered two or more times. Thus, in certain methods, the cells are contacted with between about 2 and about 50 doses of 2-carboxyaldehyde-pyridine thiosemicarbazone and ionizing radiation. Typically the cells are contacted with 2-carboxyaldehyde-pyridine thiosemicarbazone and ionizing radiation 2 to 7 times per week for between 2 and about 10 weeks, 3 to 6 times between 3 and about 8 weeks, or 4 to 5 times between about 4 and about 7 weeks. The individual dose of ionizing radiation administered to the cell is between about 0.5 Gy and about 4 Gy per dose such that the cell is contacted with an aggregate amount of radiation of between about 20 Gy and about 120 Gy, or between about 40 Gy and about 100 Gy.

In certain methods of inhibiting DNA synthesis and/or DNA repair, the 2-carboxyaldehyde pyridine thiosemicarbazone compound inhibits ribonucleotide reductase activity. More preferably, the 2-carboxyaldehyde pyridine thiosemicarbazone compound inhibits ribonucleotide reductase activity in cellular-DNA repair or DNA synthesis pathways. Certain preferred 2-carboxyaldehyde pyridine thiosemicarbazone compound inhibits ribonucleotide reductase activity in the DNA repair or DNA synthesis pathways with an IC₅₀ of less than about 10 μM, or more preferably with an IC₅₀ of less than about 5 μM.

In certain methods of inhibiting DNA synthesis and/or DNA repair, the 2-carboxyaldehyde pyridine thiosemicarbazone compound inhibits ribonucleotide reductase enzymes comprising p53R2 or hRRM2 subunits. In yet other methods the inhibitory effect of the 2-carboxyaldehyde pyridine thiosemicarbazone compound is substantially the same for ribonucleotide reductase comprising p53R2 and ribonucleotide reductase comprising hRRM2.

In certain methods of inhibiting DNA synthesis and/or DNA repair, the cells are selected from tumor cells or cancer cells. Certain suitable tumor and cancer cells include carcinoma, glioma, and neoplasms. Other suitable tumor and/or cancer cells which are suitable for use in the methods of the invention include pancreatic carcinoma, prostate carcinoma, breast carcinoma, renal carcinoma, brain gliomas, leukemia, colon cancer, and the like.

In certain methods of inhibiting DNA synthesis and/or DNA repair, the cells are selected from tumor cells or cancer cells. Certain suitable tumor and cancer cells include carcinoma, glioma, and neoplasms. Other suitable tumor and/or cancer cells which are suitable for use in the methods of the invention include pancreatic carcinoma, prostate carcinoma, breast carcinoma, renal carcinoma, and brain gliomas. Certain other suitable cancers and or tumors for treatment by the instant therapeutic methods include breast cancer, lung cancer, prostate cancer, colon cancer, ovarian cancer, cervical cancer, gastrointestinal cancer, pancreatic cancer, glioblastoma, bladder cancer, hepatoma, colorectal cancer, uterine cervical cancer, endometrial carcinoma, salivary gland carcinoma, kidney cancer, vulval cancer, thyroid cancer, hepatic carcinoma, skin cancer, melanoma, ovarian cancer, neuroblastoma, myeloma, various types of head and neck cancer, acute lymphoblastic leukemia, acute myeloid leukemia, Ewing sarcoma and peripheral neuroepithelioma, and other carcinomas, lymphomas, blastomas, sarcomas, and leukemias.

As used herein, the terms “tumor” or “cancer” refer to a condition characterized by anomalous rapid proliferation of abnormal cells in one or both breasts of a subject. The abnormal cells often are referred to as “neoplastic cells,” which are transformed cells that can form a solid tumor. The term “tumor” refers to an abnormal mass or population of cells (e.g., two or more cells) that result from excessive or abnormal cell division, whether malignant or benign, and pre-cancerous and cancerous cells. Malignant tumors are distinguished from benign growths or tumors in that, in addition to uncontrolled cellular proliferation, they can invade surrounding tissues and can metastasize. As used herein, “tumor” or “cancer,” refers to one or more of prostate, pancreas, kidney, liver, lung, brain, head and neck, mesothelioma, ovarian, urothelial, hepatocellular, bladder, esophageal, stomach, Or the like.

The term “invasion” as used herein refers to the spread of cancerous cells to adjacent surrounding tissues. The term “invasion” often is used synonymously with the term “metastasis,” which as used herein refers to a process in which cancer cells travel from one organ or tissue to another non-adjacent organ or tissue.

In another aspect of the invention, a method of treating a patient suffering from or susceptible to cancer is provided wherein the method comprises the steps of:

providing a 2-carboxyaldehyde pyridine thiosemicarbazone compound or prodrug thereof;

administering to the patient a radiosensitizing amount of the 2-carboxyaldehyde pyridine thiosemicarbazone compound or prodrug thereof; and

administering a dose of ionizing radiation e.g., in an amount or intensity sufficient to induce double strand DNA cleavage.

In certain aspects, the methods of treating a patient suffering from or susceptible to cancer comprise the administration of a 2-carboxyaldehyde pyridine thiosemicarbazone compound selected from compounds of Formula I:

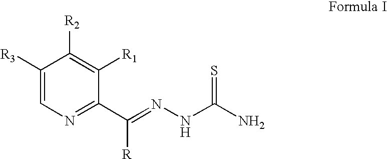

wherein

R₁ is NHR₄ or NR₄R₅, and R₃ is hydrogen; or

R₁ is hydrogen and R₃ is NHR₄, NR₄R₅ or OH;

R and R₂ are independently selected from hydrogen and C₁-C₄alkyl

R₄ is hydrogen, hydroxyl, or C₁-C₄alkyl; and

R₅ is C₁-C₄alkyl; or a pharmaceutically acceptable salt or hydrate thereof.

In certain other methods of treating a patient suffering from or susceptible to cancer, the compound of Formula I is selected from those in which R₁ is NHR₄ or NR₄R₅; and R₃ is hydrogen. In certain other methods, the compound of Formula I is selected from those compounds in which R₁ is NH₂ and R₃ is hydrogen. In certain other methods, the compound of Formula I is selected from those compounds in which R is hydrogen. In certain other methods, a compound of Formula I is administered in which R₂ is hydrogen or methyl.

In yet other methods of treating a patient suffering from or susceptible to cancer, the compound of Formula I is selected from those compounds in which R and R₂ are independently selected from hydrogen and methyl, R₁ is amino (NH₂), and R₃ is hydrogen. Certain compounds of Formula I which are suitable for use in the methods of inhibiting DNA synthesis or DNA repair include 3-amino-2-carboxyaldehyde pyridine thiosemicarbazone, 4-methyl-3-amino-2-carboxyaldehyde pyridine thiosemicarbazone, 3-amino-2-ethan-1-one-pyridine thiosemicarbazone, 4-methyl-3-amino-2-ethan-1-one pyridine thiosemicarbazone, and salts or hydrates, thereof.

In certain aspects, the methods of treating a patient suffering from or susceptible to cancer comprise the administration of a prodrug of a 2-carboxyaldehyde pyridine thiosemicatbazone compound of Formula I, e.g., a compounds of Formula II:

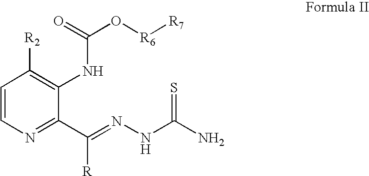

wherein

R₇ is hydrogen or methyl;

R₈ is CHR, benzyl, or ortho- or para-substituted benzyl;

R is hydrogen or C₁₋₃alkyl;

R₇ is a free acid phosphate, phosphate salt or a —S—S—R₈ residue;

R₈ is CH₂CH₂NHR₉, CH₂CH₂OH, CH₂CH₂COOR₁₀, ortho- or para-substituted phenylC₁₋₃alkyl, or ortho- or para-nitrophenyl;

R₉ is hydrogen, C₁₋₄akanoyl, trifluoroacetyl, benzoyl, or substituted benzoyl;

R₁₀ is hydrogen, C₁₋₄alkyl, phenyl, substituted phenyl, benzyl, or substituted benzyl, or a pharmaceutically acceptable salt or hydrate thereof.

Compounds of Formula II are disclosed and claimed in U.S. Pat. No. 5,767,134, which patent is incorporated by reference in its entirety.

Representative compounds of Formula I include 3-amino-2-formylpyridine thiosemicarbazone, 5-amino-2-formylpyridine thiosemicarbazone, 3-amino-4-methyl-2-formylpyridine thiosemicatbazone, 5-amino-4-methyl-2-formylpyridine thiosemicarbazone, and 5-hydroxyamino-4-methyl-2-formylpyridine thiosemicarbazone. It is to be understood that any compound of the invention above or any other aspect should be understood as contemplating any pharmaceutically acceptable prodrugs (e.g., compounds of Formula II), salts or hydrates thereof.

In certain methods of treating a patient suffering from or susceptible to cancer, the dose of ionizing radiation administered to the cell is between about 0.01 Gy to about 100 Gy, between about 0.5 Gy to about 50 Gy, or between about 1 Gy to about 20 Gy for each dose.

In certain other methods of treating a patient suffering from or susceptible to cancer, the patient is administered a protocol in which the 2-carboxyaldehyde-pyridine thiosemicarbazone compound and ionizing radiation are co-administered two or more times. Thus, in certain methods, the patient is administered with between about 2 and about 50 doses of 2-carboxyaldehyde-pyridine thiosemicarbazone and ionizing radiation. Typically the protocol comprises administering the 2-carboxyaldehyde-pyridine thiosemicarbazone compound and ionizing radiation to the patient 2 to 7 times per week for between 2 and about 10 weeks, 3 to 6 times between 3 and about 8 weeks, or 4 to 5 times between about 4 and about 7 weeks. In other methods, the protocol comprises administering the 2-carboxyaldehyde-pyridine thiosemicarbazone compound and ionizing radiation to the patient 4, 5 or 6 times per week for between 3 and about 7 weeks, or more preferably between 4 and about 6 weeks. The individual dose of ionizing radiation administered to the patient is between about 0.5 Gy and about 4 Gy per dose (or between about 1 Gy and about 3 Gy per dose) such that the aggregate amount of radiation administered to the patient is between about 40 Gy and about 120 Gy, between about 55 Gy and about 90 Gy.

In certain methods of treating cancer, a patient suffering from or susceptible to cancer, is administered a 2-carboxyaldehyde-pyridine thiosemicarbazone compound and a 2 Gy dose of ionizing radiation five times per week for about 6 weeks. Of course, the order of administration of the 2-carboxyaldehyde-pyridine thiosemicarbazone compound and ionizing radiation to the patient can be sequential or contemporaneous.

The term “co-administration” is intended to mean that ionizing radiation and a 2-carboxyaldehyde pyridine thiosemicarbazone compound are contacted with cells or administered to a patient contemporaneously or sequentially. In certain methods the cells or patients are administered the 2-carboxaldehyde-pyridine thiosemicarbazone prior to administering the ionizing radiation. In certain other methods the 2-carboxyaldehyde pyridine thiosemicarbazone compound is contacted with the cells after administering the ionizing radiation. In yet other methods, the step of contacting of 2-carboxyaldehyde pyridine thiosemicarbazone compound with the cells and the step of administering the ionizing radiation to the cells occur contemporaneously. In methods in which the 2-carboxyaldehyde-pyridine thiosemicarbazone and ionizing radiation are administered sequentially, the time period separating administration of the drug and the radiation is typically between about 15 minutes and about 48 hours, between about 15 minutes and 24 hours, or between 1 hour and 18 hours. In certain methods comprising sequential addition of the 2-carboxyaldehyde-pyridine thiosemicarbazone and ionizing radiation, the delay period between administration of the drug and radiation is about 1 hour, 2 hours, 3 hours, 4 hours, 6 hours, 8 hours, 12 hours, or 16 hours.

In certain methods of treating a patient suffering from or susceptible to cancer, it is preferable to limit the patient's exposure to ionizing radiation to that portion of the body in which the cancer or tumor is located. Thus, ionizing radiation sources which can be locally administered are generally preferred. Certain ionizing radiation sources include X-Ray sources, other high energy light sources (including gamma-ray sources and certain high energy UV light sources) and injectable radiotherapeutic agents comprising one or more radioisotopes that emit one or more high energy particles capable of double strand DNA cleavage. Therapeutic radiopharmaceuticals include those isotopes which emit Preferred radiometal ions include isotopes of metal ions that emit α, β⁻, β⁺ or γ radiation, including technetium, rhenium, yttrium, copper, gallium, indium, bismuth, platinum rhodium and iodine radioisotopes. In certain methods of the invention, X-ray irradiation is a preferred method of administering ionizing radiation to a patient.

In certain methods of treating a patient suffering from or susceptible to cancer, the 2-carboxyaldehyde pyridine thiosemicarbazone compound is administered systemically. In certain therapeutic methods, systemic delivery comprises oral delivery or intravenous injection of the 2-carboxyaldehyde pyridine thiosemicarbazone compound.

In certain other methods of treating a patient suffering from or susceptible to cancer, the 2-carboxyaldehyde pyridine thiosemicarbazone compound is delivered locally at the location of the tumor. In certain methods, the local delivery may comprise subcutaneous injection, delivery by externally guided catheterization, suppository, or the like.

In certain methods of treating a patient suffering from or susceptible to cancer, the administration of a radiosensitizing amount of the 2-carboxyaldehyde pyridine thiosemicarbazone compound and ionizing radiation to a patient suffering from or susceptible to cancer decreases tumor size by at least about 1.25 times the reduction obtained by administration of radiation alone, by at least about 1.5 times the reduction obtained by administration of radiation alone, or between about 1.5 and about 4 times reduction obtained by administration of radiation alone.

Preferred pharmaceutical compositions which are suitable for use in the methods of the present invention comprise a pharmaceutically acceptable carrier and at least one compound according to Formula I or Formula II. More preferred pharmaceutical compositions comprise 3-amino-2-carboxyaldehyde pyridine thiosemicarbazone, 4-methyl-3-amino-2-carboxyaldehyde pyridine thiosemicarbazone, or a prodrug, salt, or hydrate thereof, and a pharmaceutically acceptable carrier.

Certain preferred methods of treating patients suffering from or susceptible to cancer include treatment or prevention of cancer or other tumor disorders in mammalian patients including livestock, companion animals (dogs, cats, horses and the like), primates and humans.

Preferred methods of the invention include methods of identifying and/or selecting a subject (e.g. mammal, particularly human) that is suffering from a cancer or a growth of tumor cells and identifying compounds of Formula I or Formula II which inhibit ribonucleotide reductase enzymes which are essential to DNA synthesis or DNA repair in the cancer or growth of tumor cells.

Treatment methods of the invention include in general administration to a patient a therapeutically effective amount of one or more compounds of Formula I. In the instant therapeutic methods, a therapeutically effective amount is sufficient to radiosensitize the tumor or cancer to the detrimental effect of ionizing radiation. Suitable patients include those subjects suffering from or susceptible to (i.e. propylactic treatment) a disorder or disease identified herein. Typical patients for treatment in accordance with the invention include mammals, particularly primates, especially humans. Other suitable subjects include domesticated companion animals such as a dog, cat, horse, and the like, or a livestock animal such as cattle, pig, sheep and the like.

Preferred methods of the invention including identifying and/or selecting a subject (e.g. mammal, particularly human) that is susceptible to or suffering from a condition disclosed herein, particularly a subject that is susceptible to or suffering from one or more cancers.

A pharmaceutical composition of the invention also may be packaged together with instructions (i.e. written, such as a written sheet) for treatment of a cancer as disclosed herein, e.g. instruction for treatment of a subject that is susceptible to or suffering from cancer.

Compounds of the invention are suitably administered to a subject in a water-soluble form, e.g., as a pharmaceutically acceptable salt of an organic or inorganic acid, e.g., hydrochloride, sulfate, hemi-sulfate, phosphate, nitrate, acetate, oxalate, citrate, maleate, mesylate, etc obtained after proper chemical transformation. Also, where an acidic group is present on an inhibitor compound, a pharmaceutically acceptable salt of an organic or inorganic base can be employed such as an ammonium salt, or salt of an organic amine, or a salt of an alkali metal or alkaline earth metal such as a potassium, calcium or sodium salt. Specifically suitable pharmaceutically acceptable salts include those formed with a non-toxic cation, preferably an alkali metal cation such as K or Na, an alkaline earth metal cation such as Mg or Ca, another non-toxic metal cation such as Al or Zn or a non-toxic metalloid cation such as NH₄⁺, piperazinium or 2-hydroxyethylammonium. Certain preferred compounds suitable for use in the methods of the invention are sufficiently water soluble in neutral for such that they may be delivered without pre-generation of a pharmaceutically acceptable salt.

Compounds suitable for use in the methods of the present invention include any and all different single pure isomers and mixtures of two or more isomers. The term isomers is intended to include diastereoisomers, enantiomers, regioisomers, structural isomers, rotational isomers, tautomers, and the like. For compounds which contain one or more stereogenic centers, e.g., chiral compounds, the methods of the invention may be carried out with a enantiomerically enriched compound, a racemate, or a mixture of diastereomers. Preferred enantiomerically enriched compounds have an enantiomeric excess of 50% or more, more preferably the compound has an enantiomeric excess of 60%, 70%, 80%, 90%, 95%, 98%, or 99% or more. In certain preferred embodiments, achiral 2-carboxaldehyde-pyridine thiosemicarbazone compounds of the invention or only a single enantiomer or diastereomer of a chiral 2-carboxaldehyde-pyridine thiosemicarbazone compound is administered to a patient.

In the methods of the invention, compounds of the invention according to any one of Formula I-II and pharmaceutical compositions thereof may be administered to a subject by a variety of routes including parenteral (including intravenous, subcutaneous, intramuscular and intradermal), topical (including buccal, sublingual), oral, nasal and the like.

Compounds of the invention according to any one of Formula I-II for use in the methods of the invention can be employed, either alone or in combination with one or more other therapeutic agents, as a pharmaceutical composition in mixture with conventional excipient, i.e., pharmaceutically acceptable organic or inorganic carrier substances suitable for a desired route of administration which do not deleteriously react with the active compounds and are not deleterious to the recipient thereof. Suitable pharmaceutically acceptable carriers include but are not limited to water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with the active compounds.

For parenteral application, particularly suitable are solutions, preferably oily or aqueous solutions as well as suspensions, emulsions, or implants, including suppositories. Ampules are convenient unit dosages.

For enteral application, particularly suitable are tablets, dragees or capsules having talc and/or carbohydrate carrier binder or the like, the carrier preferably being lactose and/or corn starch and/or potato starch. A syrup, elixir or the like can be used wherein a sweetened vehicle is employed. Sustained release compositions can be formulated including those wherein the active component is protected with differentially degradable coatings, e.g., by microencapsulation, multiple coatings, etc. Tablets, capsules and syrups or other fluids are generally preferred for oral administration.

A single or combination of more than one compounds of the invention according to any one of Formulae I-II are administered in combination with ionizing radiation. In this regard, a particular therapy can be optimized by selection of an optimal 2-carboxaldehyde-pyridine thiosemicarbazone compound and optimal radiation dose, or optimal “cocktail” of therapeutic agents comprising a mixture of one or more 2-carboxaldehyde-pyridine semithiocarbazone compounds according to Formula I or Formula II, a dose of ionizing radiation, and optionally one or more additional therapeutic agents suitable for use in the treatment of cancer.

It should be understood that in addition to the ingredients particularly mentioned above the formulations of this invention may include other agents conventional in the art having regard to the type of formulation in question, for example, those suitable for oral administration may include flavoring agents.

As used herein, “alkyl” is intended to include branched, straight-chain and cyclic saturated aliphatic hydrocarbon groups including alkylene, having the specified number of carbon atoms. Examples of alkyl include, but are not limited to, methyl, ethyl, n-propyl, i-propyl, n-butyl, s-butyl, t-butyl, n-pentyl, and s-pentyl. Alkyl groups typically have 1 to about 12 carbon atoms, more typically 1 to about 8 or 1 to about 6 carbon atoms. Preferred alkyl groups are C₁₋₈ alkyl groups, more preferred are C₁₋₆-alkyl and C₁₋₄-alkyl groups. Especially preferred alkyl groups are methyl, ethyl, n-propyl and iso-propyl.

“Prodrugs” are intended to include any covalently bonded carriers which release the active parent drug according to Formula I in vivo when such prodrug is administered to a mammalian subject. Prodrugs of a compound are prepared by modifying functional groups present in the drug compound in such a way that the modifications are cleaved, either in routine manipulation or in vivo, to the parent compound. Certain preferred prodrugs of compounds of Formula I include compounds of Formula II.

Combinations of substituents and/or variables are permissible only if such combinations result in stable compounds. A stable compound or stable structure is meant to imply a compound that is sufficiently robust to survive isolation to a useful degree of purity from a reaction mixture, and formulation into an effective therapeutic agent.

In certain methods of treating a patient suffering from cancer and certain methods of inhibiting DNA synthesis activity in cells, a combination of Triapine and ionizing radiation are administered sequentially or contemporaneously to the patient or cells. The co-administration of Triapine and ionizing radiation increase cell mortality and decrease DNA synthesis activity relative to administration of either Triapine or ionizing radiation alone.

According to certain embodiments, 2-carboxyaldehyde-pyridine thiosemicarbazone (such as Triapine) may be administered in combination with other compounds, including for example, chemotherapeutic agents, anti-inflammatory agents, anti-pyretic agents radiosensitizing agents, radioprotective agents, urologic agents, anti-emetic agents, and/or anti-diarrheal agents. for example, cisplatin, carboplatin, docetaxel, paclitaxel, fluorouracil, capecitabine, gemcitabine, irinotecan, topotecan, etoposide, mitomycin, gefitinib, vincristine, vinblastine, doxorubicin, cyclophosphamide, celecoxib, rofecoxib, valdecoxib, ibuprofen, naproxen, ketoprofen, dexamethasone, prednisone, prednisolone, hydrocortisone, acetaminophen, misonidazole, amifostine, tamsulosin, phenazopyridine, ondansetron, granisetron, alosetron, palonosetron, promethazine, prochlorperazine, trimethobenzamide, aprepitant, diphenoxylate with atropine, and/or loperamide.

### EXAMPLES

It should be appreciated that the invention should not be construed to be limited to the example which is now described; rather, the invention should be construed to include any and all applications provided herein and all equivalent variations within the skill of the ordinary artisan.

Cell lines and treatment. Three human tumor cell lines were evaluated: a glioma (U251), a pancreatic carcinoma (PSN1) and a prostate carcinoma (DU145), each was obtained from the Developmental Therapeutics Program, DCTD, NCI. The cell lines were grown in RPMI 1640 (Life Technologies, Rockville, Md.) containing glutamate (5 mM) and 5% FBS and maintained at 37° C. in an atmosphere of 5% CO₂ and 95% room air. Triapine (3-amino-2-carboxaldehyde-pyridine thiosemicarbazone), provided by Vion Pharmaceuticals of New Haven, Conn., was dissolved in dimethylsulfoxide to a stock concentration of 10 mM and stored at −20° C. Cultures were irradiated using a Pantak (Solon, Ohio) X-ray source at a dose rate of 1.55 Gy/minute.

Clonogenic assay. Cell cultures were trypsinized to generate a single cell suspension and a specified number of cells were seeded into each well of 6-well tissue culture plates. In the first experiment, drug or vehicle control was, added at specified concentrations, and the plates were irradiated 16 hours later. Immediately after irradiation, the growth media was aspirated, and fresh media was added. In the second experiment, cells were seeded, and irradiated 6 hours after seeding. Immediately after irradiation, the cells were treated with the specified concentration of drug. After 16 hours of exposure, drug was aspirated, and replaced with fresh media. Ten to twelve days after seeding, colonies were stained with crystal violet, and the number of colonies containing at least 50 cells was determined and the surviving fractions were calculated. Survival curves were generated after normalizing for the cytotoxicity generated by drug alone. Data presented are the mean±SEM from at least three independent experiments. Radiation dose enhancement factors (DEFs) represent the ratio of radiation dose required to achieve 10% survival in the control cells and the drug-treated cells.

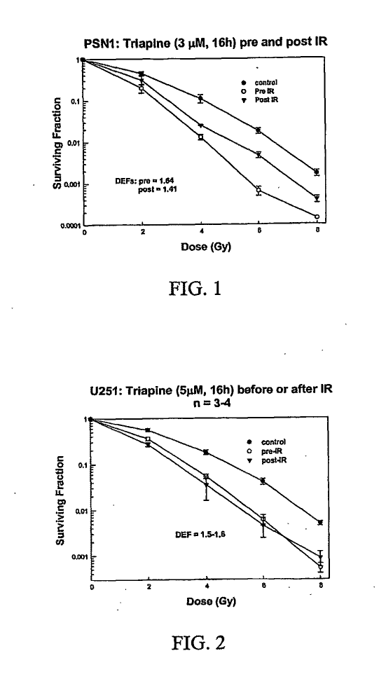

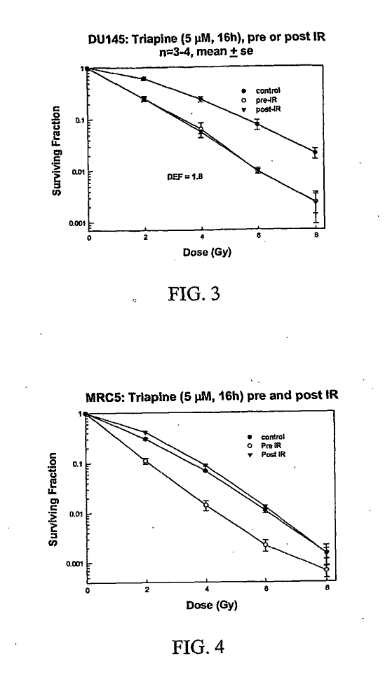

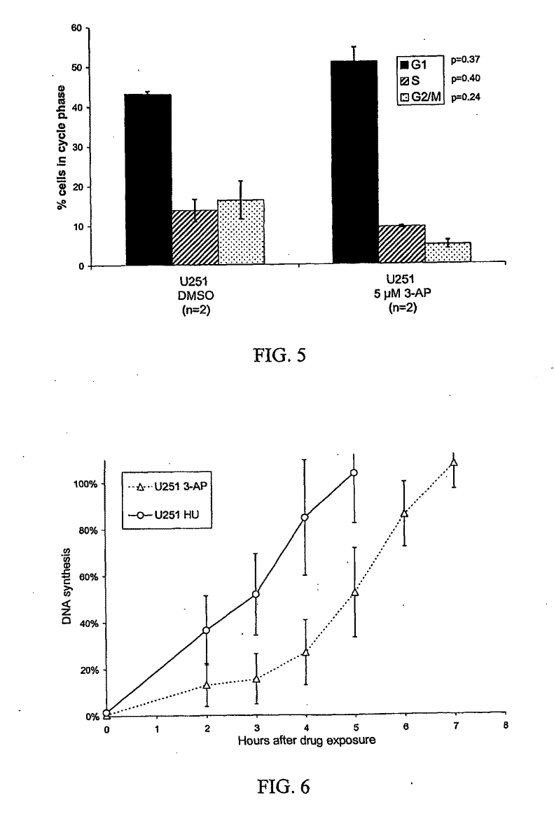

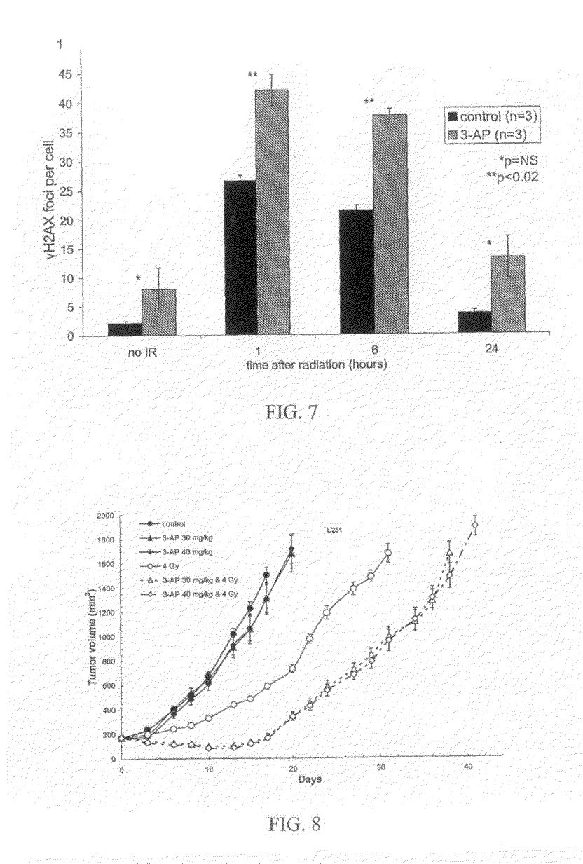

The inhibitory effect of Triapine and Triapine/radiation therapies on DNA synthesis cellular processes was assessed with a BrdU incorporation assay. A clonogenic assay assessed cell survival following drug administration before and after irradiation in a pancreatic, prostate, and glioma cell line (PSN1 (FIG. 1), DU145 (FIG. 3), and U251 (FIG. 2)). After 16 hours of Triapine exposure DNA synthesis was reduced to less than 5% of control, which returned to control levels in 7 hours (FIG. 6). Survival following 16 hours of Triapine exposure was 33% (+/−16%), 68% (+1-15%), and 53% (+1-8.3%) in the PSN1, DU145, and U251 cell lines dosed at 3, 5, and 5 μM, respectively. When Triapine was given prior to radiation, clonogenic survival revealed dose enhancement factors (DEFs) of 1.64, 2.05, and 1.50 in PSN1, DU145, and U251 cells, respectively. In combination studies, survival was normalized for the cytotoxicity induced by Triapine alone.

Cell Cycle Phase Analysis. Cell cycle phase distribution was analyzed using flow cytometry. Cells were seeded in T75 flasks at subconfluent density and exposed to Triapine, or vehicle for 16 hours, at which time cells were fixated and stained with propidium iodide for flow cytometry analysis at the Clinical Services Program at the National Cancer Institute-Frederick. A two-tailed, paired t-test was performed on the data from the replicated analysis.

Cell cycle analysis showed that 16 hours of Triapine exposure increased the percentage of cells in G1 and decreased the percentage of cells in G2/M.

To eliminate the potential contribution of cell cycle redistribution to the observed radiosensitization and as an initial test of the hypothesis that Triapine inhibits DNA repair, the cell lines were irradiated and then exposed to Triapine followed by survival analyses. Administration of Triapine for 16 hours after irradiation also resulted in radiosensitization, with the DEFs similar to those obtained when cells were exposed prior to irradiation.

DNA Synthesis Assay. DNA synthesis was quantified as an indirect measure of ribonucleotide reductase activity. A 5-bromo-2′-deoxyuridine (BrdU) incorporation assay kit (#1669915) produced by Roche Applied Science (Mannheim, Germany) was used. Cells were seeded in wells of a black wall 96-well plate and allowed to incubate for 24 hours. Drug was added, and after 16 hours of exposure, 10 μM BrdU pulses were added hourly for 2 hours, according to manufacturer's instructions. After fixation, an anti-BrdU peroxidase antibody conjugate was applied for 90 minutes at room temperature. The conjugate was then rinsed three times, and luminol was added. Chemiluminescence was detected with a Wallac 1420 multilabel counter (Perkin Elmer Life Sciences, Turku, Finland), and data was exported for off-line analysis. Triplicate samples were run for three independent experiments; data is presented as the mean of three experiments±SEM.

In Vivo Tumor Growth Delay Assay. Male 6 week old athymic nude mice (NCr nu/nu) were used in these studies. Mice, housed in filter-topped cages, were provided autoclaved feed and hyperchlorinated water ad libitum. U251 or PSN1 tumor cells were implanted subcutaneously in the hind leg. Irradiation was performed using a Pantak (Solon, Ohio) irradiator with animals restrained in a custom lead jig, which allowed for the localized irradiation of tumors at the base of the tail. When tumors reached 177 mm³ (7×7 mm) Triapine (30-60 mg/kg) was delivered by intraperitoneal injection and 6 h later tumors were irradiated with a single dose of 4 or 5 Gy. To obtain growth curves, perpendicular diameter measurements of each tumor were collected every 3 days with calipers, and the volumes were calculated using the formula (L×W×W)/2. Tumors were followed until the group's tumors were greater than 1500 mm³. Specific tumor growth delay was calculated as the number of days for the treated tumors to grow to 1400 mm³ minus the number of days for the control group to reach the same size. Each experimental group contained 10 mice and the untreated group contained 15 mice. All animal studies were conducted in accordance with the principles and procedures outlined in the U.S.P.H.S. Guide for the Care and Use of Laboratory Animals in an AAALAC-approved facility under an approved animal protocol.

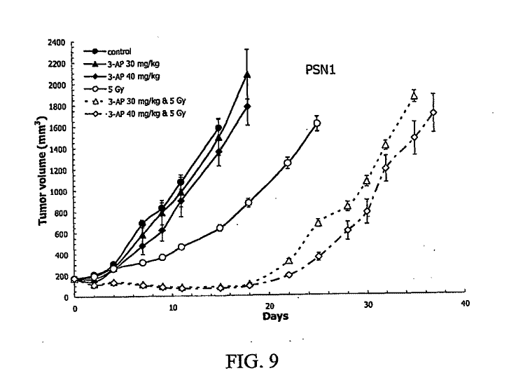

In vivo activity of Triapine in conjunction with radiation was determined through growth delays in mice with subcutaneous human tumor xenografts. Based on the above described in vitro results, xenografts of PSN1 and U251 were evaluated in vivo. Triapine (40 mg/kg) was given 6 hours prior to a single radiation dose (4 or 5 Gy). After 3 volumetric doublings of the PSN1 tumors, Triapine alone yielded a 1.6 day growth delay, and radiation caused a 9.8 day growth delay. The combination of Triapine and radiation caused a growth delay of 20.9 days, thus achieving a DEF of 2.0; a similar effect was observed in U251 tumors. See, FIG. 8 (U251) and FIG. 9 (PSN1) for a graph of tumor growth delay data.

The foregoing description of the invention is merely illustrative thereof, and it is understood that variations and modifications can be effected without departing from the spirit or scope of the invention as set forth in the following claims. Each of the documents referred to herein are incorporated by reference into the disclosure of the application.

All documents mentioned herein are incorporated herein in their entirety by reference.

1. A method for inhibiting DNA synthesis and DNA repair in a cell comprising the steps of:
providing a 2-carboxyaldehyde pyridine thiosemicarbazone compound or prodrug thereof;
contacting the cell with a radiosensitizing amount of the 2-carboxyaldehyde pyridine thiosemicarbazone compound or prodrug thereof; and
exposing the cell to ionizing radiation.

2. The method of claim 1, wherein the ionizing radiation is sufficient to induce double strand breaks in DNA of the cell.

3. The method of claim 1, wherein the cells and the compound of Formula I are contacted in vitro.

4. The method of claim 1, wherein the cells and the compound of Formula I are contacted in vivo.

5. The method of claim 1, wherein the 2-carboxyaldehyde pyridine thiosemicarbazone compound is a compound of Formula I: 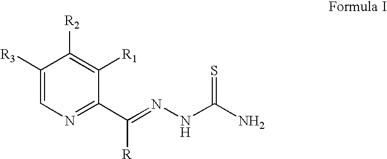
wherein
R₁ is NHR₄ or NR₄R₅, and R₃ is hydrogen; or
R₁ is hydrogen and R₃ is NHR₄, NR₄R₅ or OH;
R and R₂ are independently selected from hydrogen and C₁-C₄alkyl
R₄ is hydrogen, hydroxyl, or C₁-C₄alkyl; and
R₅ is C₁-C₄alkyl; or a pharmaceutically acceptable salt or hydrate thereof.

6. The method of claim 5 wherein R is hydrogen.

7. The method of claim 5 wherein R₁ is NHR₄ or NR₄R₅; and R₃ is hydrogen.

8. The method of claim 1 wherein the 2-carboxyaldehyde pyridine thiosemicarbazone compound is 3-amino-2-carboxyaldehyde pyridine thiosemicarbazone.

9. The method of claim 1, wherein the 2-carboxyaldehyde pyridine thiosemicarbazone compound is a compound of Formula II: 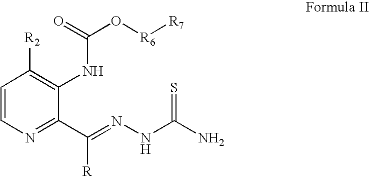
wherein
R₂ is hydrogen or methyl;
R₆ is CHR, benzyl, or ortho or para-substituted benzyl;
R is hydrogen or C₁₋₃alkyl;
R₇ is a free acid phosphate, phosphate salt or a —S—S—R₈ residue;
R₈ is CH₂CH₂NHR₉, CH₂CH₂OH, CH₂CH₂COOR₁₀, ortho- or para-substituted phenylC₁₋₃alkyl, or ortho- or para-nitrophenyl;
R₉ is hydrogen, C₁₋₄akanoyl, trifluoroacetyl, benzoyl, or substituted benzoyl;
R₁₀ is hydrogen, C₁₋₄alkyl, phenyl, substituted phenyl, benzyl, or substituted benzyl.

10. (canceled)

11. The method of claim 10, wherein the ionizing radiation is administered to the cells at a dosage of about 0.5 Gy to about 50 Gy.

12. (canceled)

13. The method of claim 1 wherein the 2-carboxyaldehyde pyridine thiosemicarbazone compound is contacted with the cells before administering the ionizing radiation.

14. The method of claim 13 wherein the 2-carboxyaldehyde pyridine thiosemicarbazone compound is contacted with the cells between about 15 minutes and about 24 hours prior to administering the ionizing radiation.

15. The method of claim 1 wherein the 2-carboxyaldehyde pyridine thiosemicarbazone compound is contacted with the cells after administering the ionizing radiation.

16. The method of claim 15, wherein the 2-carboxyaldehyde pyridine thiosemicarbazone compound is contacted with the cells between about 15 minutes and about 48 hours after administering the ionizing radiation.

17-22. (canceled)

23. The method of claim 1, wherein the cell is selected from tumor cells or cancer cells.

24-25. (canceled)

26. A method of treating a patient suffering from or susceptible to cancer, the method comprising the steps of:
providing a 2-carboxyaldehyde pyridine thiosemicarbazone compound or prodrug thereof;
administering to the patient a radiosensitizing amount of the 2-carboxyaldehyde pyridine thiosemicarbazone compound or prodrug thereof; and
administering a dose of ionizing radiation.

27. The method of claim 26, wherein the dose of ionizing radiation is sufficient to induce double strand DNA cleavage.

28. The method of claim 26, wherein the 2-carboxyaldehyde pyridine thiosemicarbazone compound is a compound of Formula I: 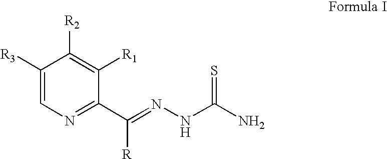
wherein
R₁ is NHR₄ or NR₄R₅, and R₃ is hydrogen; or
R₁ is hydrogen and R₃ is NHR₄, NR₄R₅ or OH;
R and R₂ are independently selected from hydrogen and C₁-C₄alkyl;
R₄ is hydrogen, hydroxyl, or C₁-C₄alkyl; and
R₅ is C₁-C₄alkyl; or a pharmaceutically acceptable salt or hydrate thereof.

29. The method of claim 26, wherein R is hydrogen.

30. The method of claim 26, wherein R₁ is NHR₄ or NR₄R₅; and R₃ is hydrogen.

31. The method of claim 26, wherein the 2-carboxyaldehyde pyridine thiosemicarbazone compound is 3-amino-2-carboxyaldehyde pyridine thiosemicarbazone.

32. The method of claim 26, wherein the 2-carboxyaldehyde pyridine thiosemicarbazone compound is a compound of Formula II: 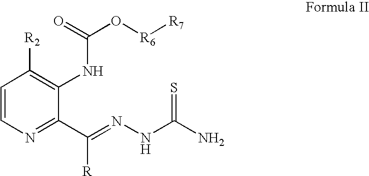
wherein
R₂ is hydrogen or methyl;
R₆ is CHR, benzyl, or ortho- or para-substituted benzyl;
R is hydrogen or C₁₋₃alkyl;
R₇ is a free acid phosphate, phosphate salt or a —S—S—R₈ residue;
R₈ is CH₂CH₂NHR₉, CH₂CH₂OH, CH₂CH₂COOR₁₀, ortho- or para-substituted phenylC₁₋₃alkyl, or ortho- or para-nitrophenyl;
R₉ is hydrogen, C₁₋₄akanoyl, trifluoroacetyl, benzoyl, or substituted benzoyl;
R₁₀ is hydrogen, C₁₋₄alkyl, phenyl, substituted phenyl, benzyl, or substituted benzyl.

33-35. (canceled)

36. The method of claim 26 wherein the 2-carboxyaldehyde pyridine thiosemicarbazone compound and ionizing radiation are administered to the patient one two or more separate occasions.

37. The method of claim 36, wherein the patient is administered doses of 2-carboxyaldehyde pyridine thiosemicarbazone compound and ionizing radiation between 2 and 7 times per week for between 2 and 10 weeks.

38-43. (canceled)

44. The method of claim 26, wherein the 2-carboxyaldehyde pyridine thiosemicarbazone compound is administered to the patient with the cells prior to administering the ionizing radiation.

45-64. (canceled)

65. The method of claim 26, wherein the patient is an mammal.

66. The method of claim 65, wherein the patient is a primate.

67. The method of claim 66, wherein the patient is a human.

68. A compound of Formula I: 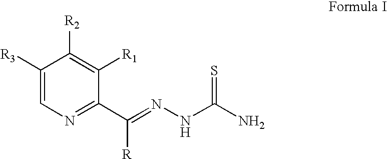
wherein
R₁ is NHR₄ or NR₄R₅, and R₃ is hydrogen; or
R₁ is hydrogen and R₃ is NHR₄, NR₄R₅ or OH;
R is C₁-C₄alkyl;
R₂ is selected from hydrogen and C₁-C₄alkyl;
R₄ is hydrogen, hydroxyl, or C₁-C₄alkyl; and
R₅ is C₁-C₄alkyl; or a pharmaceutically acceptable salt or hydrate thereof.

69. The compound of claim 68 wherein R₁ is NHR₄ or NR₄R₅; and R₃ is hydrogen.

70. The compound of claim 68, wherein the 2-carboxyaldehyde pyridine thiosemicarbazone compound is a compound of Formula II: 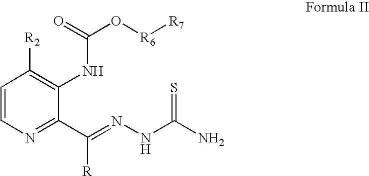
wherein
R₂ is hydrogen or methyl;
R₆ is CHR, benzyl, or ortho- or para-substituted benzyl;
R is C₁₋₃alkyl;
R₇ is a free acid phosphate, phosphate salt or a —S—S—R₈ residue;
R₈ is CH₂CH₂NHR₉, CH₂CH₂OH, CH₂CH₂COOR₁₀, ortho- or para-substituted phenylC₁₋₃alkyl, or ortho- or para-nitrophenyl;
R₉ is hydrogen, C₁₋₄akanoyl, trifluoroacetyl, benzoyl, or substituted benzoyl; R₁₀ is hydrogen, C₁₋₄alkyl, phenyl, substituted phenyl, benzyl, or substituted benzyl.
